(19) United States
(12) Patent Application Publication
(10) Pub. No.: US 2003/0207444 A1
(43) Pub. Date: Nov. 6, 2003

(54) Base sequence of initiation site for efficient protein expression in escherichia coli

(76) Inventors: Hailun Tang, North York (CA); Emil F. Pai, Toronto (CA)

Correspondence Address: Hailun Tang, 19 Heathview Avenue, North York, ON, M2K 2C2, CA

(21) Appl. No.: 10/138,309

(22) Filed: May 6, 2002

(51) Int. Cl. C12N 15/00; C07H 21/04

(52) U.S. Cl. 435/320.1; 536/23.1

(57) ABSTRACT

Base sequences of initiation sites for efficient protein expression in escherichia coli having a common base sequence, the differences between them rendered mute due to looping and truncating of the base differences

### BACKGROUND

Worldwide genome sequencing efforts have identified many genes. The identification and production of proteins, for which the genes encode, is the work of Proteomics. Proteomics urgently requires a way to express these genes as proteins, so their structure can be analyzed and elucidated.

Prokaryotic Escherichia coli, are most often used for protein expression because of their availability, robustness and well understood properties. Presently, many genes cannot be expressed using existing vectors. In some cases, this is due to the accidental occurrence of an initiation site inside the coding region of the gene destined for protein expression (internal initiation site), which interferes with the initiation site in the vector. In some cases the naturally occurring internal site is stronger than that of the vector, resulting in absent or reduced protein expression. However, in other cases the fault lies with the initiation site design of the vector.

The initiation sites of vectors have been described as having three distinct, but connected functional sections: first, the Shine Dalgarno sequence (hereinafter referred to as the “SD”); next, the spacer; and finally, the start codon “ATG” or seldom, “GTG”. The sequence of the SD is well known to the art and was first described by J. Shine and L. Dalgarno in their paper: The 3′-Terminal Sequence of Escherichia coli 16S Ribosomal RNA: Complementarity to Nonsense Triplets and Ribosome Binding Sites, Proc. Nat. Acad. Sci. USA, Vol. 71, No.4, pp. 1342-1346, April 1974. They reported that the 3′-terminal sequence of Escherichia coli 16S Ribosomal RNA is ACCUCCUUA_{OH}. The complementary sequence for mRNA is: 5′-UAAGGAGGU, and this is referred to as the SD sequence (Chen 1994). The corresponding sequence in cDNA is: TAAGGAGGT, also referred to as the SD sequence, and herein referred to as the original SD sequence. Subsequently, this cDNA sequence has been truncated and modified in many ways and incorporated into protein expression vectors. For example, the vector pET-15b (Novagen) has an SD of: AGGAG; and the vector pCRT7/NT-TOPO (Invitrogen) has an SD of AGAAGGA. The preferred embodiment of the present invention incorporates an SD of AGGAGGT, which is a subset of the original cDNA SD sequence: TAAGGAGGT—leaving out only the first two bases T and A.

As mentioned above, a spacer sequence lies between the SD and the “start codon”. Many different spacers have been used in an attempt to improve protein expression, comprised of various lengths and sequences. For example vectors pET-15b (Novagen) has a spacer length of 7 (ATATACC); and pCRT7/NT-TOPO (Invitrogen) has a spacer of 9 (GATATACAT). The fact that spacers of much larger size, 17, exist in native and functional mRNAs has raised the possibility that the base sequences contract by forming intermediary loops. (Storm, 1982). The mechanism by which this should occur is still not understood.

As mentioned above, the “start codon” that is most often used for vectors is ATG although GTG is sometimes used.

Initiation sites of gene expression, comprised of SD's, spacers and start codons, that have been previously used in vectors have proved to be inadequate. Also, this may be due to interference by the native internal initiation site in the gene.

### DETAILED DESCRIPTION OF THE INVENTION

The First Preferred Embodiment of the present invention is an initiation site in a protein expression vector that is comprised of the following sequence: AGGAGGTACCCATG.

As can be readily appreciated, this sequence is approximately a composition of an SD of AGGAGGT, a spacer of: ACCC and a start codon of: ATG. As mentioned above, the SD of the invention is therefore a truncation of the original SD sequence: TAAGGAGGT, the inventors having removed the first two bases, TA, leaving: AGGAGGT.

Studies conducted by the inventors have shown that the invention, AGGAGGTACCCATG, as an initiation site in a protein expression vector, produced large amounts of the protein encoded by the HRV2 3C gene while the vectors pET-15b (Novagen) and pCRT7/NT-TOPO (Invitrogen) produced virtually none. Other studies carried out by the inventors have demonstrated that this effect is not specific to this gene HRV2 3C and other genes have been induced to produce protein in much greater quantities using the invention as an initiation site in the inventor's protein expression vector. It should be noted that the preferred embodiments of the invention may be used as an initiation site in any vector and its application is not limited to those the inventors actually tested. Based on the inventors's studies, the preferred embodiments should improve existing vectors as well as new vectors.

As mentioned above, the spacer region of the initiation site can be composed of very long base sequences, and still be functional, raising the possibility that sections of the initiation site are looping, changing shape or altering their orientation relative to the ribosome (hereafter simply referred to collectively as “looping”), thereby shortening the length of the initiation sequence to approximate the functionality of shorter initiation sequences. The inventors have produced other preferred embodiments of the invention that include base sequences that are contained within, and are in addition to, the initiation sequence found in the First Preferred Embodiment but which they believe are looping in such a way that they are presenting only the base sequence of the First Preferred Embodiment to the ribosome and effectively truncating the initiation sequence. The inventors believe that they have discovered several rules for this looping that allow one to identify functionally equivalent initiation sequences. In order to test the loop hypothesis, the inventors specifically designed initiation sites that had the possibility of forming loops. The results of these tests were consistent with the hypothesized loops and their withdrawal from interactions with the ribosome, as well as the loops rendering the remainder of the sequence functionally equivalent to an initiation sequence that did not contain the bases that formed the loops.

The inventors believe that the sequence AGGAGGTATACCCATG, hereafter referred to as the Second Preferred Embodiment, is functionally equivalent to the First Preferred Embodiment: AGGAGGTACCCATG. The inventors believe that for TATA sequences, the middle AT bases will loop-out, functionally removing themselves from direct interaction with the Ribosome, but leaving the remainder of the base sequence to interact with the ribosome, as if it were the First Preferred Embodiment. Experiments conducted by the inventors proved that the Second Preferred Embodiment expressed protein as well as the First Preferred Embodiment, which supports the theory that they are interacting with the Ribosome in an equivalent manner. Once the looped-out AT, between the TATA sequence in the Second Preferred Embodiment AGGAGGTATACCCATG, is removed, it becomes: AGGAGGTACCCATG, the First Preferred Embodiment.

The inventors also believe that the sequence AGGAGGTACCGGCCATG, hereafter referred to as the Third Preferred Embodiment, is functionally equivalent to the First Preferred Embodiment: AGGAGGTACCGGCCATG. The inventors believe that for CCGGCC sequences, the contained bases GGC loop-out, functionally removing themselves from direct interaction with the ribosome. Experiments conducted by the inventors proved that the Third Preferred Embodiment expressed protein as well as the First Preferred Embodiment, which supports the theory that they are interacting with the ribosome in an equivalent manner.

The inventors believe that looping out may occur in sequences that have bases in addition to, and contained within, those contained in the First Preferred Embodiment, that make them functionally equivalent to the First Preferred Embodiment. It is to be understood that these initiation sites are additional preferred embodiments of this invention.

While the preferred embodiments described herein have included the start codon ATG, it is to be understood that GTG could be substituted for some additional preferred embodiments and that any reference herein, including the claims, should be read to include these additional base sequences. However, it should be noted that for most uses ATG would be the preferred start codon.

While the present invention has been described in conjunction with preferred embodiments, it is to be understood that modifications and variations may be resorted to without departing from the spirit and scope of the invention as those skilled in the art will readily understand. Such modifications and variations are considered to be within the purview and scope of the inventions and appended claims.

What is claimed is:

1. A vector initiation site for protein expression comprised of the following bases:
AGGAGGTACCCATG

2. A vector initiation site for protein expression comprised of the following bases:
AGGAGGTATACCCATG

3. A vector initiation site for protein expression comprised of the following bases:
AGGAGGTACCGGCCATG

4. The vector initiation site of claim 1, having additional base or base sequences, or both, and
the said additional base or base sequences, or both
loop, functionally truncating the base sequences, and
leaving the remaining bases to interact with the ribosome, as if such additional base or base sequences, or both, were absent or approximately absent, from the vector initiation site, of the Preferred Embodiment of claim 1.

5. The vector initiation site of claim 1, having an SD base sequence of AGGAGGT.

6. The vector initiation site of claim 1, having a spacer sequence of ACCC.

7. The vector initiation site of claim 1, having a start codon of ATG.

8. The vector initiation site of claim 1, having a start codon of GTG in substitution for the start codon ATG.

9. The vector initiation site of claim 2, having a start codon of GTG in substitution for the start codon ATG.

10. The vector initiation site of claim 3, having a start codon of GTG in substitution for the start codon ATG.
